(19) United States
(12) Patent Application Publication
(10) Pub. No.: US 2020/0361158 A1
(43) Pub. Date: Nov. 19, 2020

(54) APPARATUS FOR MANUFACTURING WEARING ARTICLE AND METHOD OF MANUFACTURING SAME

(71) Applicant: ZUIKO CORPORATION, Osaka (JP)

(72) Inventors: Masayuki SUGIURA, Osaka (JP); Takahiro SHIMADA, Osaka (JP)

(21) Appl. No.: 16/638,375

(22) PCT Filed: Sep. 10, 2018
(86) PCT No.: PCT/JP2018/033368

§ 371 Date: Feb. 11, 2020

(30) Foreign Application Priority Data
Sep. 15, 2017 (JP) 2017-177502

(51) Int. Cl. B29C 65/74; A61F 13/15; B29C 65/08; B29C 65/78; B29C 65/00

(52) U.S. Cl. CPC B29C 65/7459; A61F 13/15723; A61F 13/15764; B29C 65/08; B29L 2031/4878; B29C 65/7455; B29C 66/81433; B29C 66/43; B29C 65/7847

(57) ABSTRACT

An anvil in which a plurality of convex portions are formed on the surface of a roll, a plurality of horns that are ultrasonically vibrated to repeatedly make contact with the convex portions through an elastic sheet so as to perform melting and cutting and a plurality of vibration devices that ultrasonically vibrate the horns are included, the tip ends of the horns are arranged so as to be opposite the anvil and the horns are arranged in different positions in the axial direction of the roll so that the horns make contact with different convex portions of the convex portions.

### TECHNICAL FIELD

The present invention relates to the manufacturing of wearing articles such as disposable diapers and pants.

### BACKGROUND ART

Conventionally, a plurality of threadlike elastic members is sandwiched between two sheets of non-woven fabric, and thus a stretchable elastic sheet is formed (the first patent document). In this related art, the elastic sheet is passed between an embossing roll and an opposite roll, the embossing roll having a plurality of convex portions, and the elastic members are sandwiched together with the non-woven fabric and are cut between the convex portions and the opposite roll. In this way, in the elastic sheet, stretch regions and non-stretch regions are alternately formed.

### CITATION LIST

### Patent Literature

The first patent document: JP2002-113042 A (FIG. 1)

### SUMMARY OF INVENTION

In the related art described above, in order to reliably cut the elastic members, a pressing mechanism is needed which presses one roll to the other roll.

Moreover, in order to prevent the deformation of the roll caused by the pressing, it is necessary to increase the diameter of the rolls or to provide a side ring, with the result that the size of the entire apparatus is disadvantageously increased.

Furthermore, in order for the elastic members to be reliably cut, at least one of the rolls is heated.

Hence, an object of the present invention is to solve these problems.

The present manufacturing apparatus includes:

a production device that produces an elastic sheet in which a plurality of elastic members is sandwiched between a first sheet and a second sheet; and

a cutting device that carries out melting and cutting, by heat, a part of the plurality of elastic members in the elastic sheet,

wherein the cutting device includes:
one anvil in which convex portions are formed on a surface of a roll;
horns that ultrasonically vibrate to repeatedly contact with the convex portions through the elastic sheet so as to perform the melting and cutting; and
vibration devices that ultrasonically vibrate the horns, tip end surfaces of the horns are arranged so as to face the anvil, and

the horns are arranged in different positions in an axial direction of the roll so that the horns contact with respective different convex portions of the convex portions, wherein at least a part of each of the horns contacts with a corresponding convex portion of the different convex portions.

The “at least a part of each of the horns contacts with a corresponding convex portion of the different convex portions” means that there are two cases: one case where the horns each make contact with different convex portions without overlapping with one another; and the other case where a part of one horn and a part of another horn make contact with the same convex portion and where the remaining parts of these horns make contact with respective different convex portions.

A manufacturing method using the manufacturing apparatus described above includes:

a production step of producing the elastic sheet by sandwiching the elastic members between the first sheet and the second sheet; and

a step of cutting the elastic members by ultrasonic vibration of the horns without the first and the second sheets in the elastic sheet being melted and cut.

In the present invention, the elastic members are cut by ultrasonic vibration. Hence, the elastic members can be reliably cut. Since the cutting is performed by ultrasonic vibration, the part of the elastic sheet where the elastic members are cut looks good and is inconspicuous in a finished product. Furthermore, the part where the elastic members are cut is unlikely to be coarse, so feelings of fitting and touching the part are good.

The tip ends of the convex portions of the roll do not need to be sharp such that the convex portions are unlikely to wear. Hence, it is not necessary to polish the convex portions. Furthermore, the roll does not need to be heated, and thus it is not necessary to supply power to rotating members.

It is also not necessary to press the roll with a large force and thus a pressing mechanism is not needed, and furthermore, the diameter of the roll can be decreased and thus it is possible to reduce the size of the entire apparatus. Moreover, a side ring does not need to be provided on the roll.

Incidentally, when a single horn which is long in the axial direction of the roll makes contact with the convex portions of the roll, the contact of such horn with the roll is more likely to become nonuniform by slight inclination of the horn with the roll. By contrast, a plurality of horns are arranged in the axial direction of the roll, and thus the pressing force of each of the horns to the roll in the axial direction is uniform, with the result that the reliability of the cutting is enhanced.

### BRIEF DESCRIPTION OF DRAWINGS

FIG. 1 is a plan view in which a wearing article according to an embodiment of the present invention is unfolded and is shown.

FIG. 2 is a plan view showing the process of a method of manufacturing the wearing article.

FIG. 3A is a plan view of a cutting device and FIG. 3B is a layout view showing a manufacturing apparatus.

FIG. 4 is a schematic perspective view showing an anvil.

FIG. 5 is a layout view showing the manufacturing apparatus.

FIG. 6 is a schematic perspective view showing the anvil; and

FIGS. 7A to 7B each are schematic plan views showing examples of the arrangement pattern of horns.

### DESCRIPTION OF EMBODIMENTS

Preferably, each of the tip end surfaces of the horns extends in the axial direction of the roll along at least one of generatrices (generating lines) of the roll.

In this case, the pressing force of each of the horns to the roll in the axial direction is uniform, and thus the horns will make contact with the respective convex portions of the roll with a uniform force.

Preferably, among the horns, horns that are adjacent to each other are arranged apart from each other in the axial direction of the roll.

In this case, it is possible to prevent the adjacent horns from interfering with each other.

Preferably, the horns are arranged on a straight line along one of the generatrices of the roll.

In this case, a simple structure for supporting the horns and the vibration device will be provided.

Preferably, the horns include one horn extending along one of the generatrices and a different horn extending along a different generatrix of the generatrices that is apart from the one generatrix in a circumferential direction of the roll, and

the one horn and the different horn are arranged so as to overlap with each other, in the axial direction of the roll, only in an end portion of the one horn and an end portion of the different horn, and are arranged apart from each other in the circumferential direction of the roll.

The “overlap with each other only in an end portion of the one horn and an end portion of the different horn” means that a case where the entire horns overlap with each other in the axial direction is excluded. In other words, although one horn overlaps with other horn in their end portions in the axial direction, they do not overlap with each other in their remaining portions (most thereof) in the axial direction.

In this case, the horns can be arranged in a state where the areas to be cut by the horns overlap with each other. Hence, the reliability of the cutting the elastic members is enhanced.

Preferably, the horns include one horn extending along one of the generatrices and a different horn extending along a different generatrix that is apart from the one generatrix in a circumferential direction of the roll, and

the one horn and the different horn are arranged apart from each other in the circumferential direction of the roll, and are arranged so as not to be apart from each other in the axial direction of the roll while are arranged so as to be adjacent to each other in the axial direction of the roll.

In other words, although the one horn and the different horn are apart from each other in the circumferential direction of the roll, they are arranged adjacent to each other so that they are not apart from each other in the axial direction of the roll and do not overlap with each other.

In this case, since the horns are arranged so as to be adjacent to each other in the axial direction, the reliability of the cutting of elastic members is enhanced.

Preferably, the manufacturing apparatus includes: a vacuum device which holds the elastic sheet on the surface of the roll by suction.

Incidentally, when the multiple horns make contact with the elastic sheet, creases can easily be produced in the elastic sheet. However, it is possible to reduce the production of the creases by cutting the elastic sheet while the elastic sheet is being held by suction.

Any feature illustrated and/or depicted in conjunction with one of the aforementioned aspects or the following embodiments may be used in the same or similar form in one or more of the other aspects or other embodiments, and/or may be used in combination with, or in place of, any feature of the other aspects or embodiments.

### EMBODIMENTS

The present invention will be understood more clearly from the following description of preferred embodiments taken in conjunction with the accompanying drawings. Note however that the embodiments and the drawings are merely illustrative and should not be taken to define the scope of the present invention. The scope of the present invention shall be defined only by the appended claims. In the accompanying drawings, like reference numerals denote like components throughout the plurality of figures.

An example of the structure of a wearing article 1 according to the present invention will be described below with reference to drawings.

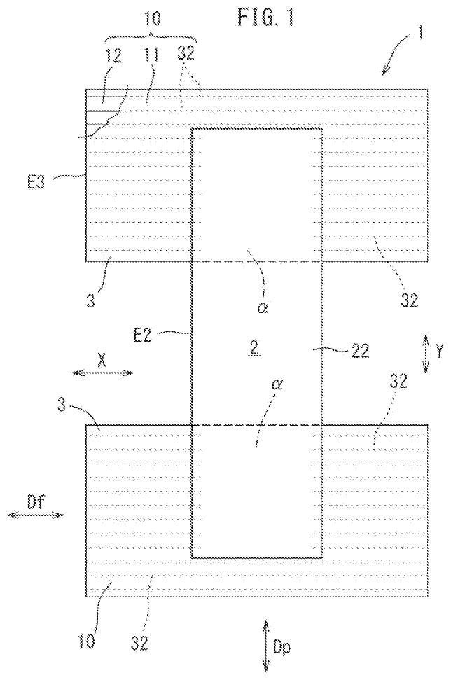

As shown in FIG. 1, a wearing article 1 has a symmetrical shape and a symmetrical structure, and includes an absorbent body 2 and front and back waist portions 3. The absorbent body 2 includes: a waist part which covers the front waist of a wearer and which is extended in a waist direction X; a waist part which covers the back waist of the wearer and which is extended in the waist direction X; and a crotch portion 22 which covers a crotch between both the waist parts.

The crotch portion 22 is extended in a longitudinal direction Y perpendicular to the waist direction X. The absorbent body 2 forms part or the whole of the crotch portion 22.

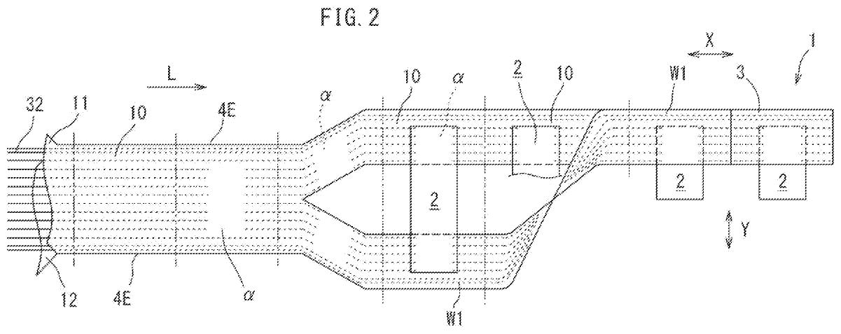

As shown in FIG. 2, in a state where the wearing article 1 is a finished product, the crotch portion 22 is folded into two in a line parallel to the waist direction X. In this way, end portions E3 of the front and back waist portions of FIG. 1 in the waist direction X are overlaid on each other. When the wearing article is a pant type, the end portions of the front waist portion 3 and the back waist portion 3 in the waist direction X are melted and adhered to each other.

An absorbent core which is not illustrated is provided in the absorbent body 2 of FIG. 1. The absorbent core absorbs body fluids. The absorbent core is sandwiched between a top sheet and a back sheet. The individual sheets and the absorbent core are stacked in layers.

The top sheet is formed of liquid permeable thin non-woven fabric, and covers the skin side of the absorbent core. On the top sheet, an unillustrated cuff may be provided.

The back sheet covers the non-skin side of the absorbent core, and is formed with a liquid impermeable resin sheet. The waist portions 3 are adhered to the end portions of the absorbent body 2 in the longitudinal direction Y.

The waist portions 3 are protruded from the absorbent body 2 in the waist direction X. In other words, the waist portions 3 are protruded beyond the crotch portion 22 in the waist direction X, and are extended out in the waist direction X from both edge portions (end portions in the waist direction) E2 of the absorbent body 2.

Elastic members 32 for fitting the wearing article 1 to the wearer are provided in each of the waist portions 3. As the elastic members 32, for example, a plurality of rubber threads, flat rubber, or a material including thermoplastic resin can be adopted. A portion of the elastic members 32 which overlaps the absorbent body 2 is disabled (brought into a state where a contraction force is not present). The individual pieces of elastic members 32 may be extended parallel to each other in the waist direction.

In the absorbent body 2, leg portions (unillustrated) which are constricted along the legs of the wearer may be formed. In the leg portions and parts of the waist portions 3 which continue to the leg portions, for example, other elastic members formed of thread rubber or the like may be provided along the legs of the wearer.

The absorbent body 2 is adhered to the skin side of the waist portions 3.

In the present specification, the “skin side” refers to a side facing the skin of the wearer when the wearing article 1 is worn, and the “non-skin side” refers to a side opposite to the skin side.

The wearing article 1 will then be further described in detail.

In FIG. 1, each of the front and back waist portions 3 is formed such that a plurality of elastic members 32 extended in the waist direction X is sandwiched between first and second sheets 11 and 12. The first and second sheets 11 and 12 are, for example, non-woven fabric pieces, and are bonded to each other with an adhesive or a melting/adhering structure so as to form an elastic sheet 10. The elastic sheet 10 forms folds (gathers) by the contraction force of the elastic members 32.

An apparatus for manufacturing the wearing article will then be described.

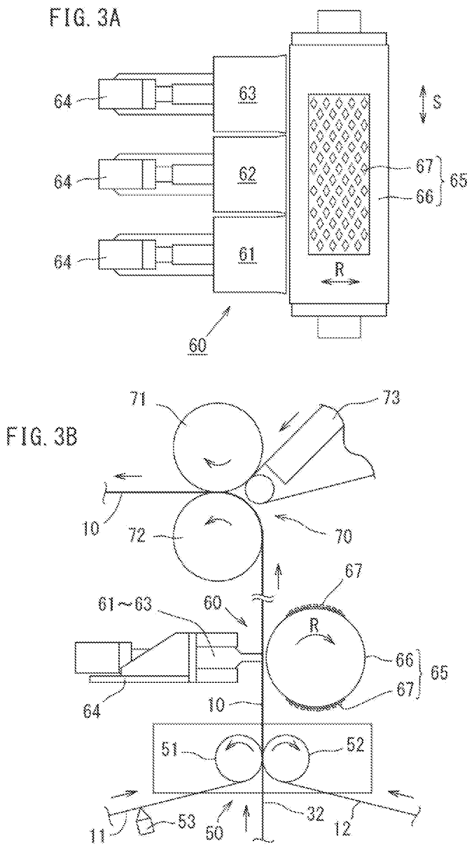

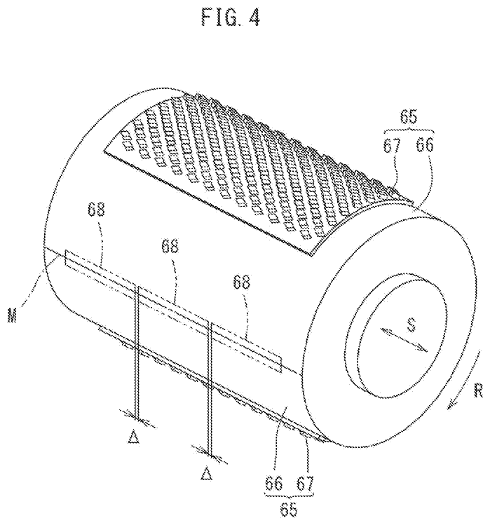

FIGS. 3A to 4 show a first embodiment.

As shown in FIG. 3B, the manufacturing apparatus includes a production device 50, a cutting device 60, a building device 70 and the like.

The production device 50 includes a pair of nip rolls 51 and 52, and uses the pair of nip rolls 51 and 52 so as to produce the elastic sheet 10 in which the elastic members 32 are sandwiched between the first sheet 11 and the second sheet 12. The first sheet 11, the second sheet 12 and the elastic members 32 are components of the produced elastic sheet 10, and are continuous members in a flow direction.

In other words, in the present embodiment, the pair of sheets 11 and 12 and the elastic members 32 are introduced into the pair of nip rolls 51 and 52, and are stacked in layers so as to produce the elastic sheet 10. An adhesive may be applied from a coating machine 53 to one of the sheets 11 (12). Alternatively, after the adhesive is applied to only the elastic members 32 without being applied to the sheet, the elastic members 32 may be sandwiched between the pair of sheets 11 and 12 so as to produce the elastic sheet.

The cutting device 60 includes, in FIG. 3A, a plurality of horns 61, 62 and 63, a vibration device 64 that is provided in each of the horns 61 to 63, and an anvil 65. The cutting device 60 melts and cuts, by heat, a part of the elastic members 32 in the elastic sheet 10 of FIG. 3B.

In the anvil 65, a plurality of convex portions 67 are formed on the surface of a roll 66. The horns 61 to 63 are ultrasonically vibrated to repeatedly make contact with the convex portions 67 through the elastic sheet 10 so as to perform the melting and cutting described above. The vibration devices 64 ultrasonically vibrate the horns 61 to 63.

As shown in FIGS. 4 and 3A, a group of the convex portions 67 in the anvil 65 is provided in part of the surface of the roll 66, and is provided in a predetermined region in a circumferential direction R and in an axial direction S of the roll 66. The convex portions 67 may be provided intermittently in the circumferential direction R and in the axial direction S or may be arranged in a staggered pattern. Regarding the number of groups of the convex portions 67, it is enough to provide at least one group in one roll 66.

As shown in FIG. 4, the tip end surfaces 68 of the horns 61 to 63 are arranged so as to be opposite the anvil 65. The horns 61 to 63 are arranged in different positions in the axial direction S of the roll 66 so as to make contact with different convex portions 67 of the convex portions 67.

In FIG. 4, the tip end surfaces 68 of the horns 61 to 63 are extended in the axial direction S of the roll 66 along the generatrix M of the roll 66. In the present embodiment, the horns 61 to 63 are arranged on a straight line along the single generatrix M of the roll 66.

Among the horns 61 to 63 (FIG. 3A), as shown in FIG. 4, the tip end surfaces 68 of the horns 61 to 63 (FIG. 3A) which are adjacent to each other are arranged only a predetermined distance Δ apart from each other in the axial direction S of the roll 66. In this way, the horns are prevented from interfering with each other.

A method of manufacturing the wearing article 1 will then be described.

As shown in FIG. 2, a plurality of continuous elastic members 32 which is extended in a transport direction L is transported in the transport direction L in a state where it is elongated in the transport direction L.

The nip rolls 51 and 52 of FIG. 3B sandwich the continuous elastic members 32 between the two continuous sheets, that is, the first and second sheets (continuous non-woven fabric) 11 and 12. The first and second sheets 11 and 12 each include a pair of side edge portions 4E that is extended in the transport direction L of FIG. 2. The nip rolls 51 and 52 bond the first and second sheets 11 and 12 together with the adhesive so that they are overlaid on each other, thereby producing the elastic sheet 10.

The bonding described above may be performed by heat seal (heat melting and adhering) or may be performed by melting and adhering with ultrasonic energy.

In parts of the elastic sheet 10 of FIG. 2 which overlap the absorbent body 2, the elastic members 32 are cut with the cutting device 60 of FIG. 3A so as to be disabled. The cutting of the elastic members 32 will be described in detail below.

As shown in FIG. 3B, the elastic sheet 10 is introduced between the horns 61 to 63 and the anvil 65. When the convex portions 67 of the anvil are rotated to such a position that the convex portions 67 are opposite the horns 61 to 63, the horns 61 to 63 make contact with the convex portions 67 by the ultrasonic vibrations of the horns 61 to 63, and thus the parts of the elastic sheet 10 which are sandwiched between the horns 61 to 63 and the convex portions 67 are increased in temperature.

On the other hand, the elastic members 32 are lower in softening point and melting point than the first and second sheets 11 and 12, and are higher in elongation during transport than them. Hence, only the elastic members 32 in the elastic sheet 10 are melted and cut by the temperature increase in a predetermined region a of FIG. 2. In other words, a step is performed in which the horns 61 to 63 are ultrasonically vibrated so as to cut only the elastic members 32 without the first and second sheets 11 and 12 of the elastic sheet 10 being melted and cut.

Thereafter, as shown in FIG. 2, the elastic sheet 10 including the pair of side edge portions 4E is slit along the transport direction L with a slitter (unillustrated) while being transported in the transport direction L. In this way, the elastic sheet 10 is formed into a pair of waist elastic sheets 10 serving as the front and back waist portions. After the slitting described above, the two waist elastic sheets 10 are relatively moved in the longitudinal direction Y perpendicular to the transport direction L so as to be apart from each other in the longitudinal direction Y.

Thereafter, while the pair of waist elastic sheets 10 is being transported substantially parallel to each other in the transport direction L, the absorbent body 2 straddles the region a in the pair of waist elastic sheets 10, and the absorbent body 2 is arranged (bridged) so as to overlap the waist elastic sheets 10, with the result that a continuous multilayer (laminate) member W1 is produced.

Specifically, the absorbent body 2 of FIG. 2 is introduced intermittently and intermittingly from the cut-out conveyor 73 of the placing device 70 in FIG. 3B into a first combine roll 71. On the other hand, through the second combine roll 72 of the placing device 70, the pair of elastic sheets 10 which has been divided is introduced between both the rolls 71 and 72. In this way, the absorbent body 2 is bridged between the pair of elastic sheets 10 by the rolls 71 and 72, and thus the continuous multilayer member W1 of FIG. 2 is produced.

Thereafter, the continuous multilayer member W1 is folded into two in the absorbent body 2 so that the pair of waist elastic sheets 10 are overlaid on each other.

As shown in FIG. 2, in the end portions E3 (FIG. 1) of the front and back waist portions 3 in the transport direction L and in side seal portions, the pair of waist elastic sheets 10 is bonded by being melted and adhered to each other. The bonding by melting and adhering may be performed by melting and adhering with the ultrasonic energy of ultrasonic horns or may be performed by heat seal.

Thereafter, the continuous multilayer member W1 of FIG. 2 is cut into the sizes (units) of individual wearing articles 1 along virtual cutting lines indicated by alternate long and short dashed lines. In other words, in order to sequentially produce the individual wearing articles 1, the continuous multilayer member W1 is sequentially cut in the longitudinal direction Y between the absorbent bodies 2 adjacent to each other in the transport direction L. Thus, in the corresponding positions to the end portions E3 of the wearing article 1 in the waist direction X (FIG. 1), pairs of waist elastic sheets 10 are sequentially cut. In this way, it is possible to obtain individual pant-type wearing articles 1.

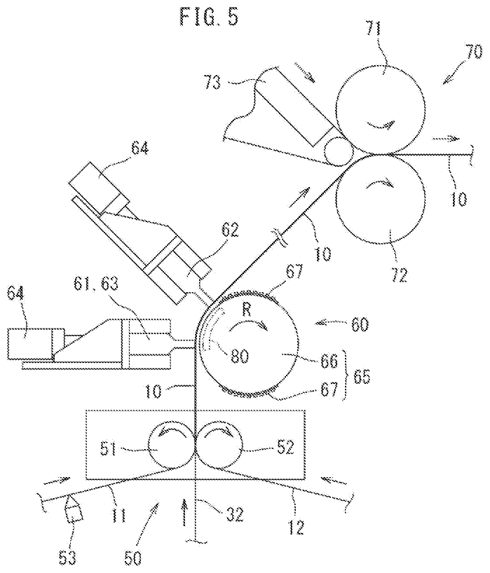

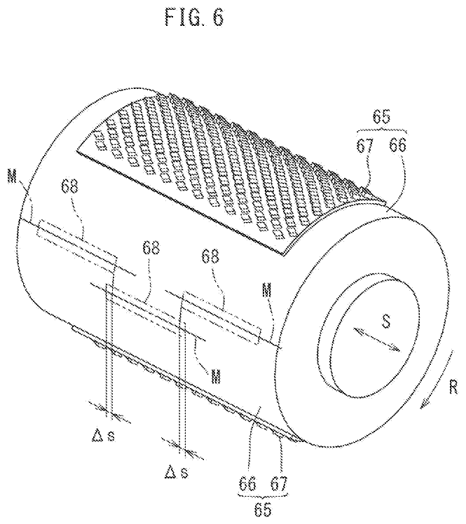

FIGS. 5 and 6 show a second embodiment.

A wearing article that is obtained in the present second embodiment is the same as in the first embodiment described above.

In the present embodiment, as in FIG. 6, the horns 61 to 63 are arranged so as to overlap with each other in the axial direction S of the roll 66 only by Δs. On the other hand, as in FIG. 5, the horns 61 and 63 and the horn 62 are arranged apart from each other in the circumferential direction R of the roll 66.

A vacuum device 80 that holds (sucks) the elastic sheet 10 on the surface of the roll 66 by suction may be provided. For example, the vacuum device 80 may hold the elastic sheet 10 on the surface of the roll 66 by suction only within a range of angles at which the horns 61 to 63 are provided.

In each of the embodiments, the anvil 65 essentially does not need to be heated, and thus a power supply for heating the anvil 65 is not provided.

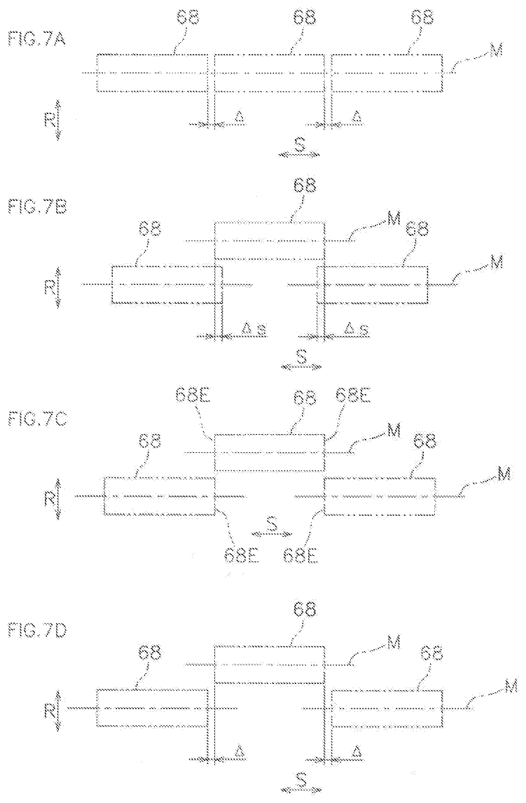

FIGS. 7A to 7D each are schematic plan views showing examples of the arrangement pattern of a plurality of horns. For ease of understanding of the drawings by simplification thereof, they are shown by use of the tip end surfaces 68 of the horns.

In the example shown in FIG. 7A, the arrangement is the same as in the first embodiment. In the present example, the horns adjacent to each other in the axial direction S of the roll are arranged apart from each other only by the predetermined distance Δ. The horns are arranged on a straight line along the single generatrix M of the roll without being displaced from each other in the circumferential direction R of the roll.

In the example shown in FIG. 7B, the arrangement is the same as in the second embodiment. In the present example, the horns include one horn (for example, each horn on left and right sides) which is extended along one generatrix M and a different horn (for example, a horn in the middle) which is extended along a different generatrix M apart from the one generatrix M in the circumferential direction R. In other words, the one horn and the different horns are apart from each other in the circumferential direction R.

On the other hand, in the axial direction S, the end portion of the one horn and the end portion of the different horns are arranged so as to overlap with each other only by Δs. In other words, most of the one horn including a center portion and most of the different horn including a center portion do not overlap with each other in the axial direction S.

In the example shown in FIG. 7C, as in the example of FIG. 7B, the horns include the one horn and the different horns. In other words, the one horn and the different horns are apart from each other in the circumferential direction R.

On the other hand, the ends 68E of the one horn and the ends 68E of the different horns are arranged so as not to be apart from each other in the axial direction S and are arranged so as to be adjacent to each other in the axial direction S. In other words, the three horns are arranged so that among the three horns aligned on a straight line without any gap in the axial direction S, only the one horn in the middle is moved in the circumferential direction R.

In the example shown in FIG. 7D, as in the example of FIG. 7B, the horns include the one horn and the different horns. In other words, the one horn and the different horns are apart from each other in the circumferential direction R.

On the other hand, the one horn and the different horns are arranged apart from each other only by Δ in the axial direction S.

Although in FIG. 7D, the one horn and the different horns do not overlap with each other in the circumferential direction R, they may be arranged so as to overlap with each other in the circumferential direction R.

Although the preferred embodiments have been described above with reference to the drawings, a person skilled in the art easily conceives various variations and modifications from the present specification in an obvious manner.

For example, the present wearing article does not need to include a cuff or the like. Even in parts other than the parts of the waist portions which overlap the absorbent body, the tension of the elastic members may be disabled.

The vacuum device may be provided in the anvil of the first embodiment.

The slitting of the elastic sheet 10 and the separation of the pair of elastic sheets 10 in the longitudinal direction Y may be performed before the elastic members 32 are cut with the cutting device 60.

Hence, the variations and modifications described above are interpreted to fall within the scope of the present invention defined from scope of claims.

### INDUSTRIAL APPLICABILITY

The present invention can be applied to wearing articles such as disposable diapers and pants.

### REFERENCE SIGNS LIST

1: wearing article, 10: elastic sheet, 11: first sheet, 12: second sheet, 2: absorbent body, 22: crotch portion, 3: waist portion, 32: elastic members, 4E: side edge portion, 50: production device, 51, 52: nip roll, 53: coating machine, 60: cutting device, 61, 62, 63: horn, 64: vibration device, 65: anvil, 66: roll, 67: convex portion, 68: tip end surface, 70: building device, 71: first combine roll, 72: second combine roll, 73: cut-out conveyor, 80: vacuum device, E2, E3: edge portion, M: generatrix, L: transport direction, R: circumferential direction, S: axial direction, X: waist direction, Y: longitudinal direction, W: continuous multilayer member, a: region

1. An apparatus for manufacturing a wearing article, the apparatus comprising:
a production device that produces an elastic sheet in which a plurality of elastic members is sandwiched between a first sheet and a second sheet; and
a cutting device that carries out melting and cutting, by heat, a part of the plurality of elastic members in the elastic sheet,
wherein the cutting device includes:
one anvil in which convex portions are formed on a surface of a roll;
horns that ultrasonically vibrate to repeatedly contact with the convex portions through the elastic sheet so as to perform the melting and cutting; and
vibration devices that ultrasonically vibrate the horns,
tip end surfaces of the horns are arranged so as to face the anvil, and
the horns are arranged in different positions in an axial direction of the roll so that the horns contact with respective different convex portions of the convex portions, wherein at least a part of each of the horns contacts with a corresponding convex portion of the different convex portions.

2. The apparatus according to claim 1,
wherein the tip end surfaces of the horns extends in the axial direction of the roll along at least one of generatrices of the roll.

3. The apparatus according to claim 2,
wherein among the horns, horns that are adjacent to each other are arranged apart from each other in the axial direction of the roll.

4. The apparatus according to claim 3,
wherein the horns are arranged on a straight line along one of the generatrices of the roll.

5. The apparatus according to claim 2,
wherein the horns include one horn extending along one of the generatrices and a different horn extending along a different generatrix of the generatrices that is apart from the one generatrix in a circumferential direction of the roll, and
the one horn and the different horn are arranged so as to overlap with each other, in the axial direction of the roll, only in an end portion of the one horn and an end portion of the different horn, and are arranged apart from each other in the circumferential direction of the roll.

6. The apparatus according to claim 2,
wherein the horns include one horn extending along one of the generatrices and a different horn extending along a different generatrix that is apart from the one generatrix in a circumferential direction of the roll, and
the one horn and the different horn are arranged apart from each other in the circumferential direction of the roll, and are arranged so as not to be apart from each other in the axial direction of the roll while are arranged so as to be adjacent to each other in the axial direction of the roll.

7. The apparatus according to claim 1, comprising:
a vacuum device that holds the elastic sheet on a surface of the roll by suction.

8. A method of manufacturing a wearing article with the apparatus of claim 1, the method comprising:
a production step of producing the elastic sheet by sandwiching the elastic members between the first sheet and the second sheet; and
a step of cutting the elastic members by ultrasonic vibration of the horns without the first and the second sheets in the elastic sheet being melted and cut.

9. The apparatus according to claim 2, comprising:
a vacuum device that holds the elastic sheet on a surface of the roll by suction.

10. The apparatus according to claim 3, comprising:
a vacuum device that holds the elastic sheet on a surface of the roll by suction.

11. The apparatus according to claim 4, comprising:
a vacuum device that holds the elastic sheet on a surface of the roll by suction.

12. The apparatus according to claim 5, comprising:
a vacuum device that holds the elastic sheet on a surface of the roll by suction.

13. The apparatus according to claim 6, comprising:
a vacuum device that holds the elastic sheet on a surface of the roll by suction.
